(19) United States
(12) Patent Application Publication
(10) Pub. No.: US 2021/0000933 A1
(43) Pub. Date: Jan. 7, 2021

(54) TUMOR VACCINE

(71) Applicant: CELL-MEDICINE, INC., Ibaraki (JP)

(72) Inventors: Masayuki NITTA, Tokyo (JP); Yoshihiro MURAGAKI, Tokyo (JP); Takashi MARUYAMA, Tokyo (JP); Tadao OHNO, Ibaraki (JP)

(73) Assignee: CELL- MEDICINE, INC., Ibaraki (JP)

(21) Appl. No.: 16/633,728

(22) PCT Filed: Jun. 20, 2019
(86) PCT No.: PCT/JP2019/024397

§ 371 Date: Jan. 24, 2020

(51) Int. Cl. A61K 39/00; A61K 41/00; A61P 35/00; A61K 38/19; A61P 37/04

(52) U.S. Cl. CPC A61K 39/0011; A61K 41/0057; A61P 37/04; A61K 38/19; A61P 35/00

(57) ABSTRACT

A tumor vaccine for use in combination with a photodynamic therapy for a malignant tumor, which contains a tumor antigen derived from a tumor tissue separated from a patient to whom the photodynamic therapy is to be applied.

### TECHNICAL FIELD

The present invention relates to a tumor vaccine. More specifically, the present invention relates to a tumor vaccine for use in combination with a photodynamic therapy.

### BACKGROUND ART

Tumors (including carcinomas) produced in the inside of the skull are generically named brain tumors, and for therapeutic treatment of glioblastoma (glioblastoma multiforme) among those, maximum extraction thereof by surgical operation, among all, has been considered effective as in the cases of the other cancers. However, it is widely known that since glioblastoma infiltratively grows in the brain, any operations will be “absolutely non-curative resection” (resulting a state that minute brain tumors remain).

For therapeutic treatment thereof, in addition to surgical resection, a postoperative radiochemical therapy using radiation irradiation and anticancer agent temozolomide (TMZ) (Stupp regimen, announced in 2005, Non-patent document 1) is still used as a standard therapy at the time point of May, 2019. However, according to the paper of Stupp, the median overall survival (mOS) was only 14.6 months, median progression-free survival (mPFS) was only 6.9 months, and two-year survival rate was only 26.5%. At the time point of May, 2019, there is no low molecular drug superior to TMZ, and even if the antibody drug, bevacizumab, having a tumor neovascularization inhibitory effect is additionally used together with the standard therapy, mOS and mPFS were only 15.7 to 16.8 months and 10.6 to 10.7 months, respectively (Non-patent documents 2 and 3). Also in the JCOG0911 INTEGRA study for newly diagnosed glioblastoma performed in this country (clinical trial in which recurrence preventing effect of interferon beta additionally used in the standard therapy was examined), mOSs were only 20.3 months and 24.0 months, and mPFSs were only 10.1 months and 8.5 months in the control group and the interferon beta administration group, respectively (Non-patent document 4). As a result, recurrences and aggravations are observed in almost all cases, and recoveries of recurrence cases were rare.

It is well known to clinicians that since glioblastoma is very intractable, and gives bad prognosis as described above, glioblastoma is one of the most malignant among malignant neoplasms, like pancreatic cancer (detection thereof tends to be too late, and therefore they become beyond medical aid, and very difficult to be cured in many cases). Therefore, it is considered that a therapy effective for glioblastoma should be similarly effective for the other malignant tumors.

Meanwhile, a photodynamic therapy is known as one of the cancer therapy methods. The homepage of the Japan Photodynamic Association (http://square.umin.ac.jp/jpa/whatPDT.html) explains that “The photodynamic therapy (henceforth it may be abbreviated as “PDT”) is a local therapy using a photochemical reaction of a photosensitive substance showing a property of accumulating in cancer induced by laser beam irradiation. PDT is a lowly invasive therapy, with which cancer lesions can be selectively treated with low energy, and which gives extremely little damage to normal tissues, unlike the conventional therapies utilizing physical destructive actions caused by laser such as photocoagulation and transpiration”, and more specifically, it is explained to be “a therapy utilizing the specific tumor tissue and neovascularized vessel accumulation property of porphyrin-related compounds and the strong cell destructing effect of singlet oxygen generated by photoexcitation” (Non-patent document 5).

Since PDT shows a strong cell destructing effect against tumor cells, it is mainly used for local therapies of cancers. In Japan, it has already been approved by the government as a therapy to which health insurance is applied, and early lung cancer, superficial esophageal cancer, early superficial gastric cancer, early uterine cervix cancer, age-related maculopathy, primary malignant brain tumor, and post-chemoradiotherapy or radiotherapy partial persistent/recurrent esophageal cancer have been approved as indications thereof. It is considered that since PDT causes the so-called immunogenic cell death so that tumor antigens are released, it induces immune responses against tumor cells (Non-patent document 6).

The inventors of the present invention have continuously attempted to apply the aforementioned PDT to brain tumor therapies. In a phase II clinical trial, a photosensitive substance, talaporphin sodium, was administered to patients beforehand at a time point of 22 to 27 hours before, a laser beam was irradiated to the residual brain tumor region at the time of resection operation of the brain tumor, and thereafter the standard therapy was performed. As a result, for 22 brain tumor cases (including 13 cases of newly diagnosed glioblastoma), a 12-month postoperative overall survival rate of 95.5%, and a 6-month progression-free survival rate of 91% could be achieved, and especially for the newly diagnosed glioblastoma cases, both the indexes were 100%. Side reactions were observed for only four cases, and all of them were mild. What is to be noted is that mOS was 24.8 months, and mPFS was 12.0 months for the glioblastoma cases, which are good results superior to those obtainable by the standard therapy (Non-patent document 7).

The cases and additional cases were further followed up for a long term, and mOS of 27.4 months, and mPFS of 19.6 months were obtained (mOS and mPFS of the control group, in which only the standard therapy was performed, and PDT was not performed, were 22.1 months and 9.0 months, respectively). There are significant statistical differences of mPFS and mOS between the cases and the control group (Non-patent document 8).

However, in the experiment of Non-patent document 7, both two of Kaplan-Meier curves for overall survival time and progression-free survival time sunk below the 50% survival rate line even within a short observation period of 32 months as the longest period, and therefore it is still difficult to treat newly diagnosed glioblastoma even by PDT. From such a viewpoint, it has been attempted to further enhance the effectiveness of PDT, and therapies based on a combination of PDT with an immunotherapy of another type as a therapy of different action principle, especially a tumor vaccine, have been examined (Non-patent document 9). However, there has not been examined so far a method of using a tumor vaccine utilizing a chemically fixed autologous tumor as an antigen in addition to PDT.

### PRIOR ART REFERENCES

### Patent documents

Patent document 1: Japanese Patent No. 5579586
Patent document 2: International Patent Publication WO2018/047797

### Non-Patent Documents

Non-patent document 1: Radiotherapy plus concomitant and adjuvant temozolomide for glioblastoma, N. Engl. J. Med., 352, pp.987-996, 2005
Non-patent document 2: Bevacizumab plus radiotherapy-temozolomide for newly diagnosed glioblastoma, N. Engl. J. Med., 370, pp.709⁻722, 2014
Non-patent document 3:A randomized trial of bevacizumab for newly diagnosed glioblastoma, N. Engl. J. Med., 370, pp.699-708, 2014
Non-patent document 4: JCOG0911 INTEGRA study: a randomized screening phase II trial of interferon beta plus temozolomide in comparison with temozolomide alone for newly diagnosed glioblastoma, J. Neurooncol., 138, pp.627-636, 2018
Non-patent document 5: Current status of photodynamic therapy in cancer treatment, http://square.umin.ac.jp/jpa/whatPDT.html
Non-patent document 6: Immunogenic cell death: can it be exploited in photodynamic therapy for cancer?, BioMed. Research International, Article ID 482160, 18 pages, 2013
Non-patent document 7: Phase II clinical study on intraoperative photodynamic therapy with talaporfin sodium and semiconductor laser in patients with malignant brain tumors, J. Neurosurg., 119, pp.845-852, 2013
Non-patent document 8: Role of photodynamic therapy using talaporfin sodium and a semiconductor laser in patients with newly diagnosed glioblastoma, J. Neurosurg., Dec 1, 1-8, 2018.doi: 10.3171/2018.7.JNS18422
Non-patent document 9: Targeting antitumor immune response for enhancing the efficacy of photodynamic therapy of cancer: Recent advances and future perspectives, Oxidative Medicine and Cellular Longevity, Article ID 5274084, 11 pages, 2016
Non-patent document 10: Clinical trial of autologous formalin fixed tumor vaccine for glioblastoma multiforme patients, Cancer Sci., 98, pp.1226-1233, 2007
Non-patent document 11: Phase I/IIa trial of fractionated radiotherapy, temozolomide, and autologous formalin-fixed tumor vaccine for newly diagnosed glioblastoma, J. Neurosurg., 121, pp.543-553, 2014

### SUMMARY OF THE INVENTION

Object to be Achieved by the Invention

An object of the present invention is to provide a means for enhancing effectiveness of a photodynamic therapy.

More specifically, the object of the present invention is to provide a means for enhancing effectiveness of a photodynamic therapy by using a tumor vaccine.

### Means for Achieving the Object

The inventors of the present invention conducted various researches in order to achieve the aforementioned object. As a result, they found that when a laser beam is irradiated on a tumor lesion in which a photosensitive substance administered to a living body beforehand accumulates to denature tumor cells, therapeutic effect for tumors of a tumor vaccine containing a tumor antigen obtained by fixing a tumor tissue derived from the living body (autologous tissue) with formalin or the like is markedly enhanced. The present invention was accomplished on the basis of the aforementioned finding.

The present invention thus provides a tumor vaccine for use in combination with a photodynamic therapy for a malignant tumor, which contains a tumor antigen derived from a tumor tissue separated from a patient to whom the photodynamic therapy is to be applied.

As preferred embodiments of the present invention, there are provided the aforementioned tumor vaccine, wherein the tumor antigen is a tumor antigen fixed with formalin; the aforementioned tumor vaccine, wherein the tumor antigen consists of microparticles prepared from a solidified tumor material selected from the group consisting of a tumor tissue, a tumor cell, and an ingredient of these; the aforementioned tumor vaccine, which contains an immunostimulant together with a fixed tumor antigen; the aforementioned tumor vaccine, wherein the immunostimulant consists of at least one kind of immunostimulant selected from the group consisting of a cytokine and a cytokine inducer; the aforementioned tumor vaccine, wherein the malignant tumor is brain tumor; the aforementioned tumor vaccine, wherein the brain tumor is glioblastoma; and the aforementioned tumor vaccine, which is for intradermal injection.

The present invention also provides a tumor vaccine for use as an adjuvant therapy agent for a photodynamic therapy for a malignant tumor, which contains a fixed tumor antigen derived from a tumor tissue separated from a patient to whom the photodynamic therapy is to be applied, and a tumor vaccine for administration to a patient to whom a photodynamic therapy for a malignant tumor is to be applied, which contains a fixed tumor antigen derived from a tumor tissue separated from the patient to whom the photodynamic therapy is to be applied.

As another aspect of the present invention, the present invention provides use of a fixed tumor antigen derived from a tumor tissue separated from a patient to whom a photodynamic therapy is applied for manufacture of the aforementioned tumor vaccine.

As further another aspect of the present invention, the present invention provides a method for therapeutic treatment, preventing recurrence, and/or inhibiting metastasis of a malignant tumor, which comprises the step of applying a photodynamic therapy to a patient having a malignant tumor, and the step of administrating a tumor vaccine containing a fixed tumor antigen derived from a tumor tissue separated from the patient to the patient.

### Effect of the Invention

By using the tumor vaccine of the present invention, which is used in combination with a photodynamic therapy, markedly higher effectiveness can be attained in therapeutic treatment, prevention of recurrence, and/or inhibition of metastasis of a malignant tumor compared with the conventional photodynamic therapy. By using the tumor vaccine of the present invention, the effect of the photodynamic therapy of a wide applicable range can be further enhanced with stimulating the immunological competence of a living body. Therefore, therapeutic treatment can be performed with an evidently higher effectiveness for glioblastoma, a highly malignant tumor for which sufficient effectiveness cannot be obtained even with the current intensive cancer therapies (refer to the examples of the present invention).

As for the action mechanism, the action of the tumor vaccine of the present invention is based on theoretically the same cell-mediated immune response irrespective of the type of tumor, and the photodynamic therapy is a physical destruction of tumor tissues by irradiation energy. Therefore, therapeutic treatment, prevention of recurrence, and/or inhibition of metastasis of a malignant tumor using the tumor vaccine of the present invention can exhibit high effectiveness against not only glioblastoma as verified in the examples of the present invention, but also other solid tumors.

### BRIEF DESCRIPTION OF THE DRAWINGS

FIG. 1 is a graph showing that the tumor vaccine of the present invention can directly act on antigen-presenting cells to promote production of cytokine TNFa, which was shown in vitro.

FIG. 2 is a graph showing that, in the group of cases where the patients were subjected to a photodynamic therapy at the time of the surgical resection in addition to the standard therapy (Stupp regimen), and intradermally administered the tumor vaccine of the present invention, long-term survivors increased, and median overall survival was not reached in observation over 40.6 months at the longest.

### MODES FOR CARRYING OUT THE INVENTION

The photodynamic therapy is a local therapy method utilizing a photochemical reaction of a photosensitive substance showing a property of accumulating in cancer (for example, porphyrin, derivatives thereof, etc.) induced by laser beam irradiation as explained in the homep age of the Japan Photodynamic Association or Non-patent document 5, and is widely used as a standard therapy method for cancer therapy. The photodynamic therapy as the object of the application of the tumor vaccine of the present invention is not particularly limited, and the conditions for the therapy such as type and dose of the photosensitive agent, and type and irradiation intensity of the laser beam can be arbitrarily chosen. Although the photodynamic therapy is performed for solid cancers, metastatic cancers may be the object of the application in addition to primary cancers.

Although the tumor vaccine of the present invention can be administered in one or two of periods before, during, or after the photodynamic therapy, it is generally preferable to administer it after the photodynamic therapy with an interval, for example, several days to several weeks.

The tumor vaccine of the present invention can be used in combination with any one or two of standard therapies for cancer therapy, such as surgical operation, cancer chemotherapy, radiotherapy, and cancer immunotherapy. For example, a method of extracting a malignant tumor by surgical operation, then performing a photodynamic therapy with performing a standard chemotherapy as required, and thereafter administering the tumor vaccine of the present invention, a method of performing a radiotherapy, performing a photodynamic therapy with performing a standard chemotherapy as required, and then administering the tumor vaccine of the present invention, and the like can be exemplified. It is also possible to combine it with, for example, a treatment with an immune checkpoint inhibitor as a cancer immunotherapy. However, the other therapies that can be combined are not limited to these. Regimen and type of chemotherapic drug for performing chemotherapy can of course be arbitrarily selected.

Examples of the solid cancer as a target of photodynamic therapy include, for example, skin cancer, melanoma, kidney cancer, gastric cancer, lung cancer, liver cancer, breast cancer, uterine cancer, pancreatic cancer, brain tumor, and the like, but are not limited to these. Although glioblastoma, which is an intractable brain tumor, is an appropriate object for application of the tumor vaccine of the present invention, the object is not limited to glioblastoma.

The tumor vaccine of the present invention is a tumor vaccine for use in combination with a photodynamic therapy for a malignant tumor, and is characterized by containing a fixed tumor antigen derived from a tumor tissue separated from a patient to whom the photodynamic therapy is to be applied. The tumor vaccine of the present invention preferably contains an immunostimulant. The tumor antigen is preferably a tumor antigen fixed with formalin, and preferably consists of microparticles prepared from a solidified tumor material selected from the group consisting of a tumor tissue, tumor cells, and an ingredient of these. Alternatively, a dissolution product prepared from a solidified tumor material selected from the group consisting of a tumor tissue, tumor cells, and an ingredient of these can also be used as the tumor antigen.

The tumor vaccine of the present invention preferably contains an immunostimulant. The immunity is roughly divided into natural immunity and acquired immunity, and a substance that induces inflammatory reactions in a living body within several minutes to several days mainly induces natural immune responses. After the natural immune responses, acquired immunity is induced, and responses specific to a substance used as an antigen occur. The responses are mainly humoral immune responses (antibody production) caused by B cells and cell-mediated immune responses (removal of abnormal cell damage) caused by T cells. An antigen to be administered to a living body for stimulating immunity and thereby preventing (or treating, as the case may be) a disease such as infectious disease is the vaccine. A substance to be added for assisting the stimulation of immunity and enhancing immune responses against an antigen when the immunity-stimulating ability of the antigen expected to induce immune responses is weak (antigenicity is low) is the immunoadjuvant. In contrast, an agent to be singly administered without antigen for enhancing immunological competence of a living body is the “biological response modifier (henceforth abbreviated as BRM)”.

The term immunostimulant used in this specification means not only an agent that shows a direct stimulating action for specific immune responses against a specific antigen, but also an agent that shows a stimulating action also for non-antigen specific immune responses irrespective of the presence or absence of an antigen other than such an antigen as mentioned above. As described above, the “immunostimulant” includes (a) “immunoadjuvant” used together with an antigen and expected to enhance an immune response specific to the antigen, and (b) BRM to be used without an antigen for general activation of the immunological competence. Further, (b) BRM includes (b-1) “immune response enhancer” that directly stimulates an immune response process even in a partial manner, and (b-2) “immune response suppressing action inhibitor” that indirectly stimulates immunity conversely by an action of inhibiting an immune response suppressing action based on inhibition of an immune response process.

The tumor vaccine of the present invention preferably contains a cytokine as an immunostimulant, and in such a case, one or two or more kinds of cytokines may be used. For example, TNFa is preferred, and IFNg that can promote the production of TNFa is also preferred. Further, it is preferable to use granulocyte-macrophage colony-stimulating factor (henceforth abbreviated as GM-CSF), and it is also preferable to use GM-CSF and IFNg in combination. An immunostimulant that can stimulate immunocompetent cells in a local site of the body to eventually realize the same condition as that obtainable by administration of TNFa, GM-CSF, and/or IFNg, i.e., a cytokine inducer, can also be used, but the immunostimulant is not limited to these.

These cytokine and cytokine inducer are preferably prepared as a sustained release preparation so that concentration thereof at an administration site can be maintained to be high for a period as long as possible. Various means for such sustained release are known in this industry, and any of such means may be used.

The tumor vaccine of the present invention may contain an immunoadjuvant that induces a nonspecific immune response. One kind of adjuvant may be used, or two or more kinds of adjuvants may be used in combination. Examples of the adjuvant include, for example, bacteria preparations such as Freund complete adjuvant, Freund Incomplete adjuvant and BCG, bacterial ingredient preparation such as tuberculin, keyhole limpet hemocyanin, natural polymer substances such as yeast mannan, alum, TiterMax Gold, polyinosinic-polycytidylic acid stabilized with polylysine and carboxymethylcellulose (poly-ICLC), and synthetic adjuvant preparations such as a synthetic oligonucleotide containing CpG motives (CpG-ODN), and the like, but are not limited to these examples, and any substance that has an effect as an adjuvant may be used. Whether an adjuvant is used or not can be judged by referring to strength of the inflammatory responses at the local administration site, or strength of antitumor effect induced as a result of the administration as indexes. For example, it is also possible to alternately administer a tumor vaccine containing an adjuvant and a tumor vaccine not containing any adjuvant at the same site.

For example, a tumor vaccine that consists of a composition containing a formalin-solidified tumor tissue and/or tumor cells of a patient and/or a solidified fragment derived from a dissolution product of the fore goings as an antigen, and an immunoadjuvant as an immunostimulant, and can be administered to the patient himself or herself for the purpose of the therapy of tumor remaining in the body of the patient (Patent document 1, Non-patent document 10) is a preferred example of the tumor vaccine of the present invention. For example, a phase II early clinical trial was performed for one example of the aforementioned tumor vaccine, in which the vaccine was used in addition to a radiochemotherapy after surgical extraction of newly diagnosed glioblastoma, and such favorable results as mOS of 22.2 months, two-year survival rate of 47%, and three-year survival rate of 38% were obtained (Non-patent document 11). This vaccine is one of particularly preferred embodiments of the tumor vaccine of the present invention, but the tumor vaccine of the present invention is not limited to this specific tumor vaccine.

As the tumor vaccine of the present invention, a tumor vaccine in the form of a composition consisting of a combination of the aforementioned tumor antigen and at least one kind of cytokine and/or a cytokine inducer may be administered to a patient, or the aforementioned tumor antigen and at least one kind of cytokine and/or cytokine inducer may be separately administered to a patient.

As the tumor tissue or tumor cells for the preparation of the tumor vaccine of the present invention, any kind of tumor tissue or tumor cells may be used, so long as, for example, they are tissue or cells containing a tumor antigen of a tumor as a target of therapeutic or prophylactic treatment, and separated from a patient who is the target of a photodynamic therapy (autologous tissue or cells), and the type thereof is not particularly limited. When an ingredient of a tumor tissue or tumor cells is used, the type thereof is not limited so long as it contains a substance that can serve as a tumor antigen. A biological sample containing tumor cells and separated or collected from a living body, such as solid cancer tissue, bone marrow, leucocyte fraction of peripheral blood, and cell fraction of ascites or pleural effusion, can be used as a tumor material. As such an ingredient of a tumor tissue or tumor cells as mentioned above, for example, antigen proteins and antigen peptides can be used. The tumor material is generally obtained by extraction of cancer based on surgical operation, or biopsy.

The fixing method for preparing the solidified tumor material is not particularly limited, and any means available for those skilled in the art may be employed. For example, when a chemical tissue fixing agent is used, formalin, glutaraldehyde, alcohols such as methanol and ethanol, and the like may be used, but other than these, any method may be used so long as a method enabling solidification of a biological tissue, cells or an ingredient thereof is chosen. The tumor material may be solidified by such a method as paraffin embedding and freezing. When a tissue that is originally in a solid state such as an osseous tissue is used as a solidified tumor material, it is also desirable to fix it by an appropriate method.

The method for preparing the microparticles is not particularly limited, and for example, a method of grinding a solidified tumor tissue to prepare microp articles as fine fragments thereof, as well as a method of dissolving fragments obtained by grinding a tumor tissue or tumor cells, and fixing the resultant on solid microparticles, a method of fixing a soluble tumor antigen such as antigen peptide and antigen protein on solid microparticles, and the like can be employed. When a dissolution product prepared from a solidified tumor material is used as a material, for example, it can be mixed with serum albumin that is easily coagulated by thermal denaturation, or the like, then they can be uniformly mixed and coagulated by thermal denaturation to enroll the dissolution product in the coagulation product, and the coagulated product can be fragmented to be prepared in the form of microparticles. As the solid microp articles, for example, iron powder, charcoal powder, polystyrene beads, and the like having a diameter of about 0.05 to 1000μm can be used. Fragments obtained by grinding a tumor tissue, tumor cells, or soluble tumor antigen bound to lipid particles such as liposomes so that they can be recognized as microparticles and englobed by antigen-presenting cells, and a soluble tumor antigen itself made into microparticles by binding the molecules thereof with one another with a binder or cross-linking agent may also be used.

Although size of the microp articles is not particularly limited, it is desirably such a size that cells having a phagocytic ability can englobe them in the body. Tumor cells originally in the form of single separate small cells are not particularly required to be ground, but when they are coagulated by the cell fixation operation, it is desirable to subject them to grinding or dispersing treatment. As such grinding or dispersing treatment, homogenizer treatment, ultrasonication, partial digestion with a digestive enzyme, and the like can be used. The microparticles can also be prepared by passing the material through a mesh having a pore size not larger than 1000 gm, preferably not larger than 380 gm. These methods for preparing microparticles are well known to those skilled in the art, and those skilled in the art can prepare the microparticles by using any appropriate single method or combination of two or more kinds of the methods.

As the method for preparing a dissolution product from a solidified tumor material, for example, a method of using a proteolytic enzyme can be employed. Examples of the proteolytic enzyme include, for example, pronase K. A method of using an appropriate combination of an enzyme other than proteolytic enzyme, acid or alkali, and the like may also be used. Any type of method may be used so long as a method that enables dissolution of a solidified tumor material is chosen, and those skilled in the art can choose an appropriate method. The dissolution product may be fixed on the aforementioned solid microparticles.

The term “dissolution product” used in this specification means a dispersion comprising an aqueous medium such as water, physiological saline or buffer in which a solidified tumor material is dispersed to such an extent that any solid is not visually observed in the aqueous medium, and the dispersoid can be englobed by antigen-presenting cells, but the term should not be construed in any limitative way. Since the details of the method for preparing a solidified tumor material and the method for preparing microparticles are specifically described the examples of Patent document 1, those skilled in the art can prepare desired microparticles by referring to the aforementioned general explanations and specific explanations of the examples with adding appropriate modifications to these methods as required. The immunity-stimulating action of the tumor vaccine produced from dissolved fixed tumor cells is disclosed in Patent document 1 as a cytotoxic T cell induction effect.

Although the preparation form of the tumor vaccine of the present invention is not particularly limited, it is desirably in a preparation form suitable for local administration. The method for preparing the preparation is not also particularly limited, and a preparation in a desired form can be prepared by using any single kind of method or an appropriate combination of methods among the methods available in this industry. For preparing the preparation, aqueous medium such as distilled water for injection and physiological saline, and one or more kinds of pharmaceutical additives available in this industry can be used. For example, buffering agent, pH modifier, dissolving aid, stabilizer, soothing agent, preservative, and the like can be used, and specific ingredients of these are well known to those skilled in the art. The tumor vaccine can also be prepared as a solid preparation such as lyophilized preparation, and a suspending solvent such as distilled water for injection can be added to it before use to prepare an injection.

When a photodynamic therapy is performed by using the tumor vaccine of the present invention, the tumor vaccine may be administered only one time, but it is desirable to repetitively administer it to the same local site in the body so that the tumor antigen and a cytokine or cytokine inducer coexist as long as possible. For example, it is desirable that such a condition that inflammatory reactions are induced at the local site of the administration, and immunocytes concentrate and are retained there is attained. When the tumor vaccine not containing any adjuvant is administered, an adjuvant may be administered at the same local site. The tumor vaccine can generally be administered to a patient from whom the tumor material has been derived, but the tumor vaccine can also be administered to a patient with a tumor containing a tumor antigen of the same species as or close species to the tumor antigen contained in the tumor material as determined on the basis of pathological diagnosis.

Although the local site for the administration is not particularly limited, for example, it is particularly preferably injected into the skin where a material administered does not easily disperse and disappear, and many antigen-presenting cells (Langerhans cells) reside (intradermal injection). It is also preferable to inject it into a tumor tissue denatured after PDT (in situ injection), and the both type of injections may be combined. Further, it is also preferably administered into a site under the skin, or in a muscle, lymph gland or major organ such as spleen, where a cytokine and the like does not easily diffuse and disappear. However, the administration at an arbitrary site may be made possible by choosing such a dosage form that the active ingredients of the tumor vaccine do not easily diffuse, and even systemic administration may be made possible by applying a drug delivery system. Although dose and administration period of the tumor vaccine of the present invention are not particularly limited, it is desirable to appropriately determine the dose and administration period by confirming the effect of the therapy with the vaccine.

Although the tumor vaccine of the present invention is generally administered to a patient himself or herself from whom the tumor material for the preparation of the tumor vaccine has been derived (autologous administration), even a tumor vaccine prepared from a tumor material of another patient may be regarded as a tumor vaccine containing an autologous tumor antigen and administered (allogenic administration), when it can be rationally presumed to contain a tumor antigen commonly contained in the autologous tumor. Such a scheme is of course fall within the scope of the present invention. When the tumor vaccine contains a cytokine inducer, the tumor vaccine also exhibits a tumor antigen-nonspecific immunity-stimulating action (action as BRM), and therefore when a vaccine prepared from an allogenic tumor material is used, the vaccine preferably contains a cytokine inducer.

### EXAMPLES

Hereafter, the present invention will be still more specifically explained with reference to examples. However, the scope of the present invention is not limited by the following examples.

The terms and concepts used for the explanations of the present invention are based on the meanings thereof conventionally used in this field, and the techniques used for implementing the present invention can be easily and surely carried out by those skilled in the art on the basis of descriptions of published literatures etc. except for the techniques of which sources are specifically indicated. Various kinds of analyses and the like were performed according to the methods described in the instructions, catalogues, and the like of the analytical instruments, reagents, and kits used.

TABLE 1
Abbreviation table

Abbreviation

| | |
|---|---|
| 3% medium | RPMI 1640 culture medium containing 3% |
| | heat-treated fetal bovine serum |
| AFTV | Tumor vaccine of the present invention |
| BCG | Bacille de Calmette et Guérin |
| BRM | Biological response modifier |
| CpG-ODN | Synthetic oligonucleotide containing CpG motif |
| ELISA | Enzyme-linked immunosorbent assay |
| GM-CSF | Granulocyte-macrophage colony-stimulating factor |
| IFNg | Interferon gamma |
| KPS | Karnofsky performance status |
| LPS | Lipopolysaccharide |
| mOS | Median overall survival |
| mPFS | Median progression-free survival |
| PBS | Ca⁺- and Mg⁺-free phosphate buffer of Dulbecco |
| PDT | Photodynamic therapy |
| PBS-HSA | PBS containing 0.01% human serum albumin |
| PMA | Phorbol 12-myristate 13-acetate |
| TMZ | Temozolomide |
| TNFa | Tumor necrosis factor alpha |

### Example 1: Cytokine-Inducing Action of Tumor Vaccine of the Present Invention

As one of the characteristics of the tumor vaccine of the present invention, it was confirmed by the following test that it can directly stimulate cytokine production by immunocompetent cells.

It is known that if differentiation of human macrophage-like cell strain THP-1 cells is induced with phorbol 12-myristate 13-acetate (PMA) during culture thereof, they not only show a phagocytic ability, but also acquire an antigen-presenting ability, and become antigen-presenting cells. Further, if the cells are pretreated with human IFNg, which is a cytokine, the antigen-presenting ability thereof to T cells is enhanced. Differentiated THP-1 cells having shown phagocytosis produce TNFa. Production of TNFa indicates activation of macrophages (or antigen-presenting cells), and it is known that activation of macrophages (or antigen-presenting cells) in the inside of the body is the starting point of the following inflammatory reactions and immune responses (Patent document 2). Therefore, if differentiation of THP-1 cells into antigen-presenting cells is induced, then the tumor vaccine of the present invention is added to the culture medium, and amount of TNFa produced is measured, the inflammatory reaction and immune response-stimulating action of the tumor vaccine in the inside of the body can be measured in a cell culture system outside of the body.

### (1) Method for Preparing Tumor Vaccine of the Present Invention Used as Sample 1

By using a glioblastoma tissue extracted from a glioblastoma patient (hospital was Tokyo Women's Medical University Hospital, patient's clinical record number was 26254593, operation day was December 26, 2014), and subjected to treatments of from conventional formalin fixation to blocking by paraffin embedding as a starting material, an autologous tumor vaccine was prepared according to the method described in Non-patent document 10.

The endotoxin contents of the sample 1 and a 2-fold concentration liquid thereof were lower than the detection limit (0.5 EU/mL, 0.05 ng/mL in terms of lipopolysaccharide) as determined by the LAL test method using Kinetic-QCL 192 Test Kit (Lonza).

### (2) Preparation of Lipopolysaccharide (Henceforth Abbreviated as LPS) Standard Solutions

By using Dulbecco's phosphate buffered saline (PBS, calcium and magnesium-free) containing 0.01% human serum albumin (henceforth referred to as PBS-HSA), LPS (Sigma-Aldrich, L4516⁻1MG) was dissolved at a concentration of 1 mg/mL. The solution was diluted to the specified LPS concentrations with the same PBS-HSA.

### (3) Method for Bioassay of Amount of TNFa Produced by Antigen-Presenting Cells

Density of human macrophage-like cell strain THP-1 cells being cultured as maintenance culture in a conventional manner was adjusted to 500,000 cells/mL with a culture medium. The culture medium was RPMI 1640 culture medium containing 3% fetal bovine serum heat-treated in a conventional manner (henceforth referred to as 3% medium). To this cell suspension, a PMA solution (solution prepared by dissolving PMA of SIGMA in dimethyl sulfoxide at a concentration of 1.62 mM) was added at a final concentration of 0.16 μM, and the cells were inoculated by putting 0.5 mL per well of the suspension into wells of a 24-well plate, and cultured for 4 days to induce differentiation of the THP-1 cells into antigen-presenting cells.

After the culture for inducing differentiation, the medium was exchanged with a culture medium for assay (3% medium containing 0.016 μM PMA and 0.5 ng/mL of human interferon gamma (hIFNg)), and the culture was continued overnight. Then, the medium was exchanged with fresh culture medium for assay, the following sample solutions were each added to 0.5 mL per well of the culture medium for assay, and the cells were cultured for 22 hours (this culture is henceforth referred to as “assay culture”).
Sample 1: Tumor vaccine of the present invention, (preparation in the form of suspension), 32μL
Sample 2: PBS-HSA, 32 μL (TNFa production amount observed with this sample was used as the blank value, LPS concentration was 0 ng/mL)
Sample 3: PBS-HSA containing LPS (0.125 ng/mL), 32 μL
Sample 4: PBS-HSA containing LPS (0.25 ng/mL), 32 μL
Sample 5: PBS-HSA containing LPS (0.5 ng/mL), 32 μL

The culture medium obtained after the assay culture was centrifuged on a refrigerated microcentrifuge at 4° C. and 12000 rpm (maximum acceleration was 11000 G) for 5 minutes, the supernatant was collected, and diluted with 3% medium 2 times for the supernatant obtained with the sample 1, or 10 times for the supernatants obtained with the other samples, and the amount of produced TNFa was measured by enzyme-linked immunosorbent assay (ELISA). The experiment was performed in 2 or 3 wells for one sample, and average values were used as the data.

### (4) Method for Measuring TNFa

A commercially available TNFa measurement kit based on the ELISA method (OptEIA Set Human TNF, BD Biosciences, Cat. No. 555212) was used. The details of the ELISA method were according to the instruction attached to this kit. Since the concentrations of the standard TNF contained in this kit and the final color intensity of the color development reaction (A450 value obtained by using a widely used commercial TMB substrate solution and 2 N H2504 reaction termination solution) show very good linear relationship, the A450 values obtained by the plate reader as they are, but from which the blank value obtained with the sample 1 was subtracted, are used for the graph as the final data.

### (5) Results

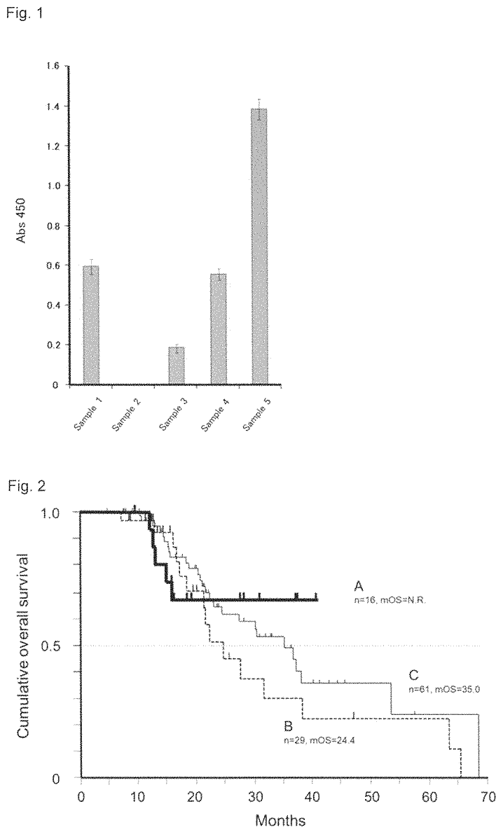

The results of the TNFa production amount are shown in FIG. 1. The values were those obtained after subtraction of the blank value (obtained with the sample 2). The results show that the tumor vaccine of the present invention can directly stimulate the production of the cytokine TNFa by antigen-presenting cells.

The fact that cell-mediated immune responses can be stimulated in vivo by administration of an autologous tumor vaccine prepared with a glioblastoma tissue derived from a patient himself or herself (that is, activation of immunocompetent cells is induced via antigen-presenting cells in the body) has been demonstrated by the delayed-type hypersensitivity response described in Non-patent document 11.

### Example 2: Enhancement of Effect of PDT for Glioblastoma with Tumor Vaccine of the Present Iinvention

### (1) Objective Patients

The newly diagnosed glioblastoma patients subjected to conventional brain tumor extraction in the Tokyo Women's Medical College, Neurosurgery Division (henceforth referred to as this hospital) in the period of from April, 2009 to December, 2016 were used as the objects (PDT was used for the brain tumor treatment from April, 2009 to December, 2010 as clinical trial, and from January, 2014 as medical service under health insurance). For the cases for which informed consents were obtained beforehand, PDT was performed during the operation in this hospital. Thereafter, the standard therapy according to the Stupp regimen (Non-patent document 1) was performed for all the cases. For the cases in which an additional treatment with the tumor vaccine of the present invention (henceforth referred to as AFTV) was hoped, AFTV was additionally administered in related medical facilities of this hospital, not in this hospital, on patients' own expense, since AFTV was a drug not approved yet by the government.

Therefore, the patients can be divided into the following three groups.
(A) PDT-received and AFTV-administered group
(B) PDT-received group
(C) AFTV-administered group

All are treatments chosen by the patients within the range of ordinary medical examination, and the results mentioned later are based on backward analysis. The treatments do not constitute a positive clinical trial in which objective patients are narrowed down beforehand, and therapies are set for the patients before the start of treatments.

Since it has already been clarified that recurrence of glioblastoma is strongly suppressed, and hence therapeutic effect is increased in the additional PDT treatment group compared with the standard therapy according to Stupp regimen alone group (Non-patent document 7), analysis was not performed for the standard therapy alone group in this example.

### (2) Background Factors of Objective Patients

The background factors of the objective patients are shown in Table 2.

TABLE 2

| | | | Group | | |
|---|---|---|---|---|---|
| | | | A | B | C |
| | | | PDT + AFTV | PDT | AFTV |
| | | Number of cases | 16 | 29 | 61 |
| | | Average age ± SD | 51.4 ± 14.4 | 54.1 ± 14.1 | 52.8 ± 14.5 |
| | | Male/female | 10/6 | 14/15 | 35/26 |
| KPS before operation | | Median | 70% | 70% | 90% |
| | | Range | 50 to 80% | 60 to 80% | 40 to 100% |
| | Number of | 70% or higher | 13 | 22 | 53 |
| | cases | Lower than 70% | 2 | 5 | 2 |
| | | Unknown | 1 | 2 | 6 |

(KPS: Karnofsky performance status)

Except for the 11 cases of the group B as the initially chosen objective patients of PDT, there was basically no difference of background factors of the patients among the patients of all the other cases, although they were cases found by diagnosis performed in routine medical care, provided that the median of KPS was slightly higher in the group C, and there was no significant bias in the selection of the patients.

### (3) PDT Treatment Protocol for Brain Tumor and Standard Therapy of Glioblastoma According to Stupp Regimen

### Day Before Operation Day

At the time considered to be 20 to 24 hours before the laser irradiation, talaporfin sodium (Laserphyrin, Meiji Seika Pharma) was administered by intravenous injection at a dose of 40 mg/m². Then, light shielding of the patients was started.

### Operation Day

After completion of the extraction of brain tumor by craniotomy, the extraction cavity wall was irradiated with a semiconductor laser (Panasonic Healthcare, medical equipment marketing approval number 22700BZX00165000). The irradiation time for 1 time was 180 seconds, and the radiation unit was automatically turned off in 180 seconds. The wavelength of the laser beam was 664 nm, and the irradiation area had a round shape having a diameter of 15 mm. The radiation power density was 150 mW/cm², and the radiation energy density was 27 J/cm².

The irradiation was performed perpendicularly to the extraction cavity wall as much as possible. As for the irradiation distance, the irradiation was performed with such a distance that two of the guide lights crosses at the irradiation object.

The irradiation was performed in a state that there was neither cerebrospinal fluid nor blood on the irradiation site as much as possible.

Attention should be paid so that blood vessels were not directly irradiated.

The number of times of the irradiation was determined depending on the size of the extraction cavity, so that irradiation areas should not overlap with one another.

### Postoperative Care

After the operation, hospitalization of the patients was continued under light shielding, and a photosensitivity test was performed one week later. Specifically, a hand covered with a thick glove having a hole of an about 2 cm size was directly exposed to sunlight for about 5 minutes, and if there was no rubor, light shielding was cancelled, and if there was rubor, light shielding was further continued. Within the postoperative 3 days and 2 weeks after the operation, diagnostic imaging of the brain was performed by MRI in a conventional manner, and presence or absence of complications such as dropsy and bleeding in the circumference of the irradiation site was confirmed.

Thereafter, the standard therapy according to the Stupp regimen was performed (Non-patent document 1). In this therapy, an accompanying treatment consisting of radiation irradiation and temozolomide administration was performed for contiguous 6 weeks, and a maintenance treatment using temozolomide was intermittently performed after temporally leaving the hospital. Although the period of the maintenance treatment was 6 months in principle, it might be different for each patient in consideration of the patient's conditions.

### (4) AFTV Treatment Protocol

The treatment with AFTV was performed according to the method described in Non-patent document 11. As for the number of times of the administration, the administration was performed for one course (AFTV was intradermally injected once per week, 3 times in total) in principle. The injection was not performed in the temozolomide administration period, and performed in drug withdrawal periods during the maintenance therapy in principle.

### (5) Results

Kaplan-Meier curves of the cumulative overall survivals obtained for the case groups are shown in FIG. 2. In the period after 12 months to around 24 months from the operation, there was almost no difference in the survival rate among the groups A, B, and C, and it gradually decreased. However, after 24 months, evident differences were observed. In particular, whereas the median overall survivals (mOS) were 24.4 months and 35.0 months in the groups B and C, respectively, in the group A, death was not observed in the 16th month and thereafter, long term survivors increased, and mOS was not reached (N.R.), and thus the group A showed extremely favorable treatment results.

Although the longest observation period of the group A was as short as 40.6 months (it still exceeded the longest PDT treatment observation period of 32 months used in Non-patent document 7), survival longer than 24 months was observed for 6 cases out of 16 cases (38%), and it can be construed that the results shown in FIG. 2 are stable. The three-year survival rate of the group A can be read to be 67% in FIG. 2. However, the three-year survival rate of the groups B and C were 30% and 49%, respectively, and the group A showed clearly higher three-year survival rate.

Therefore, it can be seen that, according to the aforementioned method for therapeutic treatment effective for glioblastoma, which is considered to be most malignant among human malignant neoplasms, in the case of therapeutic treatment after surgical extraction of another type solid tumor of equivalent or lower malignancy, a photodynamic therapy can be performed for a tumor extraction site where there are highly possibly remaining tumor cells, and an autologous tumor vaccine can be intradermally injected in a period that is not an anticancer agent administration period according to a standard therapy for that tumor.

### INDUSTRIAL APPLICABILITY

If the tumor vaccine of the present invention is used in a combination therapy with a photodynamic therapy, it can markedly enhance the therapeutic effectiveness against malignant tumor, and provides clearly higher therapeutic effect even for glioblastoma considered most malignant. Therefore, it is useful for therapeutic treatment, suppression of recurrence, and prevention of metastasis of various kinds of solid malignant tumors.

1. A tumor vaccine for use in combination with a photodynamic therapy for a malignant tumor, which contains a tumor antigen derived from a tumor tissue separated from a patient to whom the photodynamic therapy is to be applied.

2. The tumor vaccine according to claim 1, wherein the tumor antigen is a tumor antigen fixed with formalin.

3. The tumor vaccine according to claim 1, which contains an immunostimulant together with a fixed tumor antigen.

4. The tumor vaccine according to claim 1, wherein the immunostimulant consists of at least one kind of immunostimulant selected from the group consisting of a cytokine and a cytokine inducer.

5. The tumor vaccine according to claim 1, wherein the malignant tumor is glioblastoma.
